(19) United States
(12) Patent Application Publication
(10) Pub. No.: US 2014/0180109 A1
(43) Pub. Date: Jun. 26, 2014

(54) Ultrasound Servicing System and Method

(71) Applicant: GENERAL ELECTRIC COMPAN, Schenectady, NY (US)

(72) Inventors: Leif Schmedling, Toensberg (NO); Morgan Jakobsen, Horten (NO); Svein Arne Aase, Trondheim (NO)

(73) Assignee: GENERAL ELECTRIC COMPANY, Schenectady, NY (US)

(21) Appl. No.: 13/721,260

(22) Filed: Dec. 20, 2012

(51) Int. Cl. A61B 8/00; A61B 8/14

(52) U.S. Cl. CPC A61B 8/582; A61B 8/145
USPC 600/447

(57) ABSTRACT

An ultrasound servicing system is disclosed. The ultrasound servicing system comprises a near field communication enabled ultrasound scanner, and a near field communication enabled mobile device, wherein when the device is brought within a near field communication range of the scanner, a near field communication link is established for servicing the ultrasound scanner.

### BACKGROUND OF THE INVENTION

Ultrasound imaging systems require service at regular intervals. Generally, the service session consists of manual operations performed by a service technician. These operations include but are not limited to, for example, downloading software updates to the system and uploading system report files.

Generally, to obtain service access on the ultrasound scanner, a physical token or device is often required. Once service access is obtained, data files are transferred using USB memory sticks. This kind of manual operation can be time consuming and prone to human error.

The use of wireless technology in hospitals is often frowned upon for several reasons. Wireless communication traditionally requires manual configuration and is less secure than a hard-wired connection. Additionally, wireless technology can interfere with other hospital equipment. For these and other reasons, an improved ultrasound servicing system and method is desired.

### BRIEF DESCRIPTION OF THE INVENTION

The above-mentioned shortcomings, disadvantages and problems are addressed herein which will be understood by reading and understanding the following specification.

In an embodiment, an ultrasound servicing system is provided that includes a near field communication enabled ultrasound scanner enabled to communicate with a near field communication enabled mobile device when the device is brought within a near field communication range.

In an embodiment, an ultrasound servicing system is provided that includes a near field communication enabled ultrasound scanner, and a near field communication enabled mobile device, wherein when the device is brought within a near field communication range of the scanner, a near field communication link is established for servicing the ultrasound scanner.

In another embodiment, a method of servicing an ultrasound scanner comprises positioning a near field communication enabled mobile device in a near field communication range to a near field communication enabled ultrasound scanner. When the mobile device and scanner are within range, a near field communication link is established. The mobile device and/or user is authenticated and the ultrasound scanner is serviced via the near field communication link.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment of the disclosure;

FIG. 2 is a flow chart illustrating a method in accordance with an embodiment of the disclosure; and

FIG. 3 is a flow chart illustrating a method in accordance with an embodiment of the disclosure.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

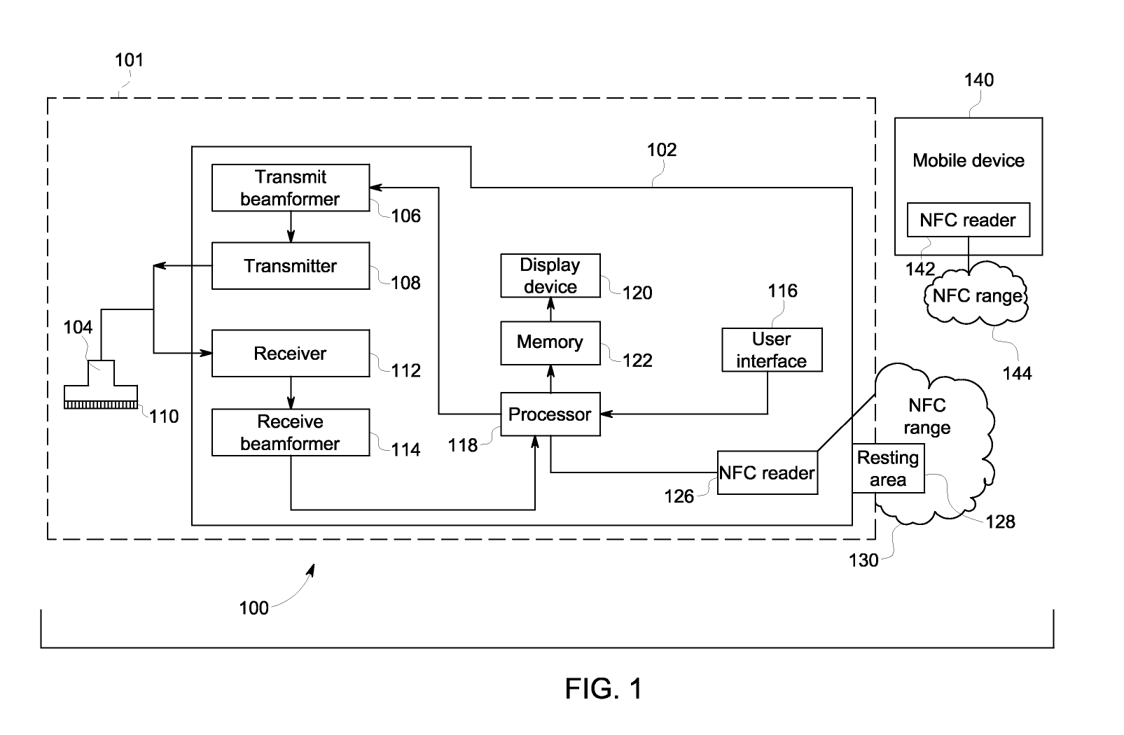

FIG. 1 is a schematic diagram of an ultrasound servicing system 100 in accordance with an embodiment of the disclosure. The ultrasound servicing system 100 may comprise an ultrasound imaging system 101 and a mobile device 140. The ultrasound imaging system 101 is generally known to those skilled in the art. The ultrasound imaging system 101 may comprise ultrasound scanner 102 and probe 104. The ultrasound scanner 102 may include a transmit beamformer 106 and a transmitter 108 that drive elements 110 within the probe 104 to emit pulsed ultrasonic signals into a body (not shown). The probe 104 may be an 2D array probe according to an embodiment. However, any other type of probe that is fully steerable in an elevation direction and capable of acquiring four-dimensional (4D) ultrasound data may be used according to other embodiments. For purposes of this disclosure, the term four-dimensional ultrasound data, or 4D ultrasound data, is defined to include ultrasound data including multiple volumes of a region-of-interest acquired over a period of time. The 4D ultrasound data contains information about how a volume changes over time. Each of the volumes may include a plurality of 2D images or slices.

Still referring to FIG. 1, the pulsed ultrasonic signals are back-scattered from structures in the body, such as blood cells or muscular tissue, to produce echoes that return to the elements 110. The echoes are converted into electrical signals, or ultrasound data, by the elements 110 and the electrical signals are received by a receiver 112. The electrical signals representing the received echoes are passed through a receive beamformer 114 that outputs ultrasound data. According to some embodiments, the probe 104 may contain electronic circuitry to do all or part of the transmit and/or the receive beamforming. For example, all or part of the transmit beamformer 106, the transmitter 108, the receiver 112 and the receive beamformer 114 may be situated within the probe 104. The terms “scan” or “scanning” may also be used in this disclosure to refer to acquiring data through the process of transmitting and receiving ultrasonic signals. The terms “data” or “ultrasound data” may be used in this disclosure to refer to either one or more datasets acquired with an ultrasound imaging system. A user interface 116 may be used to control operation of the ultrasound imaging system 101, including, to control the input of patient data, to change a scanning or display parameter. and the like.

The ultrasound imaging system 101 also includes a processor 118 to control the transmit beamformer 106, the transmitter 108, the receiver 112 and the receive beamformer 114. The processor 118 is in electronic communication with the probe 104. The processor 118 may control the probe 104 to acquire data. The processor 118 controls which of the elements 110 are active and the shape of a beam emitted from the probe 104. The processor 118 is also in electronic communication with a display device 120, and the processor 118 may process the data into images for display on the display device 120. For purposes of this disclosure, the term “electronic communication” may be defined to include both wired and wireless connections.

The processor 118 may include a central processor (CPU) according to an embodiment. According to other embodiments, the processor 118 may include other electronic components capable of carrying out processing functions, such as a digital signal processor, a field-programmable gate array (FPGA) or a graphic board. According to other embodiments, the processor 118 may include multiple electronic components capable of carrying out processing functions. For example, the processor 118 may include two or more electronic components selected from a list of electronic components including: a central processor, a digital signal processor, a field-programmable gate array, and a graphic board. According to another embodiment, the processor 118 may also include a complex demodulator (not shown) that demodulates the RE data and generates raw data. In another embodiment the demodulation can be carried out earlier in the processing chain.

The processor 118 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For example, an embodiment may acquire and display data a real-time volume-rate of 7-20 volumes/sec. However, it should be understood that the real-time frame rate may be dependent on the length of time that it takes to acquire each volume of data. Accordingly, when acquiring a relatively large volume of data, the real-time volume-rate may be slower. Thus, some embodiments may have real-time volume-rates that are considerably faster than 20 volumes/sec while other embodiments may have real-time volume-rates slower than 7 volumes/sec. The data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, 10 Hz to 30 Hz. Images generated from the data may be refreshed at a similar volume-rate. Other embodiments may acquire and display data at different rates. For example, some embodiments may acquire data at a volume-rate of less than 10 Hz or greater than 30 Hz depending on the size of the volume and the intended application. A memory 122 is included for storing processed frames of acquired data. In an exemplary embodiment, the memory 122 is of sufficient capacity to store at least several seconds worth of frames of ultrasound data. The frames of data are stored in a manner to facilitate retrieval thereof according to its order or time of acquisition. The memory 122 may comprise any known data storage medium.

In various embodiments of the present invention, data may be processed by other or different mode-related modules by the processor 118 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

The ultrasound scanner 102 also comprises a near field communication (hereinafter abbreviated “NFC”) reader 126. NFC is a short range wireless communication protocol that has been standardized in ISO/IEC 18092/ECMA-340 and ISO/IEC 21481/ECMA-352. As understood by those skilled in the art, the NFC reader 126 is adapted to emit a small electric current, which creates a magnetic field that defines a NFC range 130. The NFC range 130 extends up to a distance of about 20 cm from the NFC reader 126. It should be appreciated, however that the NFC range may extend less than 20 cm. For example, in one embodiment, the NFC range extends about 10 cm, while in another embodiment the NFC range extends 4 cm. It should also be appreciated that the NFC activation field may extend to more than 20 cm so long as the field can be generated by a NFC protocol. The NFC reader 126 may be adapted to read information, send information, or both. As such, the NFC reader 126 may be capable of receiving the magnetic field from another NFC-enabled device to communicate data or other information.

Ultrasound servicing system 100 may comprise a mobile device 140. The mobile device 140 may be, for example, a phone, a tablet or a portable computer; however, mobile device 140 should not be limited to those examples, as other types of mobile devices may be envisioned. Mobile device 140 comprises a NFC reader 142. Similar to NFC reader 126, the NFC reader 142 is adapted to emit a small electric current, which creates a magnetic field that defines a NFC range 144. Similar to NFC range 130, the NFC range 144 extends up to a distance of about 20 cm from the NFC reader 142. It should be appreciated, however that the NFC range may extend less than 20 cm. For example, in one embodiment, the NFC range extends about 10 cm, while in another embodiment the NFC range extends 4 cm. It should also be appreciated that the NFC activation field may extend to more than 20 cm so long as the field can be generated by a NFC protocol. The NFC reader 142 may be adapted to read information, send information, or both. As such, the NFC reader 142 may be capable of receiving the magnetic field from another NFC-enabled device to communicate data or other information.

Mobile device 140 may also comprise an application capable of supporting service tasks. These service tasks include updating software, transferring scanner configurations, collecting diagnostic data, and triggering test routines.

The ultrasound scanner may comprise a resting area 128 on the outer housing of the scanner 102. The resting area 128 is located in the NFC range 130 to aid in positioning the mobile device 140 relative to the scanner 102. Since the NFC range 130 is relatively small and invisible, it is helpful to the technician to indicate the location of the NFC reader within the scanner housing that creates the NFC range. In an exemplary embodiment, the resting area 128 comprises a platform. In another embodiment, the resting area 128 comprises a holder shaped to receive the mobile device 140. It should be appreciated these are simply exemplary embodiments of the resting area 128 and that other embodiments may be envisioned.

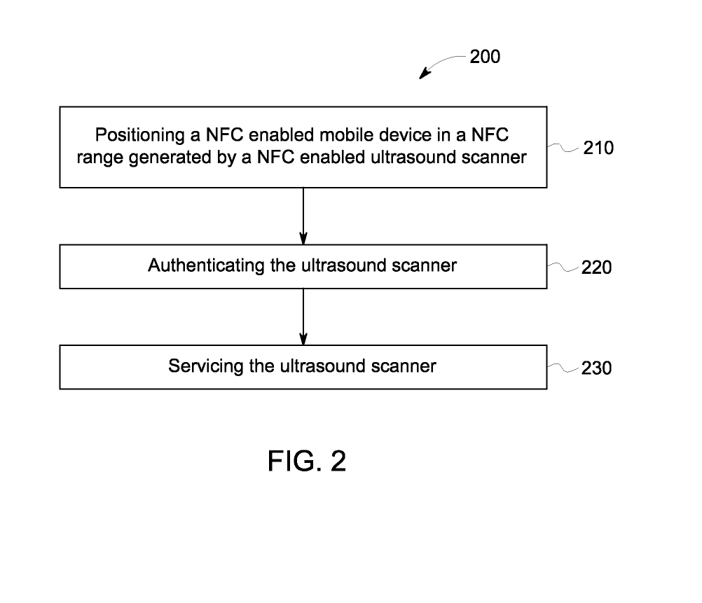

Having described the exemplary components of the system 100, a method for servicing the system 100 will now be described. An exemplary method for servicing an ultrasound scanner is generally depicted in FIG. 2. The method 200 may include positioning a NFC enabled mobile device in a NFC range to a NFC enabled ultrasound scanner wherein a NFC link is established in step 210. In an embodiment, step 210 may be accomplished by the technician holding the mobile device in the NFC range 130. In another embodiment, the mobile device 140 may be positioned in the resting area indicated on the scanner, which is within the NFC range 130.

The method 200 may also include step 220 wherein, via the NFC link, the ultrasound scanner and the mobile device are authenticated relative to each other. In an embodiment, the authentication step 220 may be challenge-response supported whereby the technician may be prompted to provide a password or pin code. In another embodiment, the authentication step is nearly instantaneous as the scanner is able to recognize the technician's identifying information from mobile device 140.

The method 200 may also include servicing the ultrasound scanner via the NFC link in step 230. In one embodiment, the mobile device 140 may be capable of running an application to service the ultrasound scanner. The mobile device application may be capable of running a library of predefined, standardized service test routines. In another embodiment, the technician is able to access a service mode of the ultrasound scanner and manually begin the service tasks. Servicing the ultrasound scanner may comprise at least one of updating software, transferring scanner configurations, collecting diagnostic data, and triggering test routines. Scanner configurations may include specially tuned or improved configuration profiles.

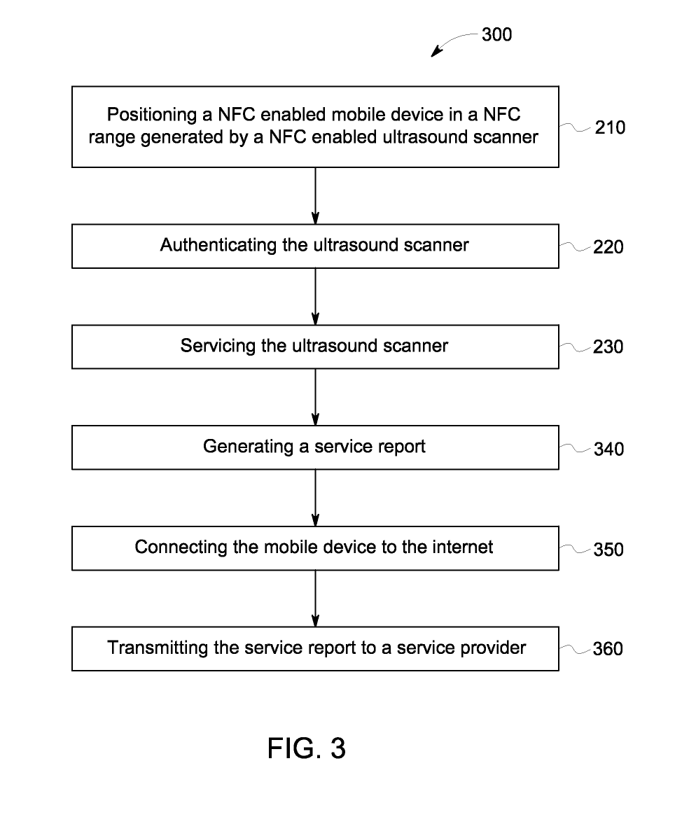

An exemplary method for servicing an ultrasound scanner is generally depicted in FIG. 3. The method 300 includes steps 210, 220, and 230 as described hereinabove with reference to FIG. 2. The method 300 may further include generating a service report in step 340. For example, in an embodiment, as mobile device 140 runs standardized test routines, the results may be grouped and tabulated into a service report. In another embodiment, the scanner 102, the mobile device 140, or both are able to produce a summary of the services performed on the scanner 102.

The method 300 may further include connecting the mobile device to the internet in step 350. The mobile device 140 may use higher bandwidth wireless protocols such as Bluetooth or Wi-Fi to connect to the internet.

The method 300 may further include step 360 which comprises transmitting the service report generated in step 340 to a service provider. In an embodiment, the service report is transmitted to the scanner manufacturer. In another embodiment, the service report may be transmitted to a third party. It should be appreciated, however, that other types of service providers may be envisioned.

As described in various embodiments herein, the NFC-enabled ultrasound service system will enable ultrasound equipment to support a service workflow that will feel natural to the service technician. With this invention service technicians will become more efficient and effective, being able to perform their tasks in a less error prone way. This will allow service providers to provide customers with affordable high quality service.

NFC, while being a wireless technology, provides advantages in comparison to conventional wireless technologies such as Bluetooth and Wi-Fi. NFC communicates over a short rage, typically less than 20 cm, which greatly decreases the possibility of security breach. Additionally, the short range emits signals with much lower power than conventional wireless technologies, and therefore decreases the chances of interfering with other equipment.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An ultrasound servicing system comprising:
a near field communication enabled ultrasound scanner enabled to communicate with a near field communication enabled mobile device when the device is brought within a near field communication range.

2. The ultrasound servicing system of claim 1, wherein the near field communication range has a radius of less than 20 cm.

3. The ultrasound servicing system of claim 1, wherein the ultrasound scanner comprises a near field communication reader.

4. The ultrasound servicing system of claim 1, wherein the ultrasound scanner comprises a resting area located within the near field communication range.

5. An ultrasound servicing system comprising:
a near field communication enabled ultrasound scanner; and
a near field communication enabled mobile device, wherein when the device is brought within a near field communication range of the scanner, a near field communication link is established for servicing the ultrasound scanner utilizing the mobile device.

6. The ultrasound servicing system of claim 5, wherein the ultrasound scanner comprises a near field communication reader.

7. The ultrasound servicing system of claim 5, wherein the mobile device comprises a near field communication reader.

8. The ultrasound servicing system of claim 5, wherein the near field communication range has a radius of less than 20 cm.

9. The ultrasound servicing system of claim 5, wherein the ultrasound scanner comprises a mobile device resting area located within the near field communication range.

10. The ultrasound servicing system of claim 5, wherein the ultrasound scanner is capable of providing identification information to the mobile device.

11. The ultrasound servicing system of claim 5, wherein the mobile device is capable of providing service actions to the ultrasound scanner.

12. The ultrasound servicing system of claim 11, wherein the service actions comprise at least one of updating software, transferring scanner configurations, collecting diagnostic data and triggering test routines.

13. The ultrasound servicing system of claim 5, wherein the mobile device is capable of generating a service report.

14. The ultrasound servicing system of claim 13, wherein the mobile device is capable of transmitting the service report to a service provider database.

15. The ultrasound servicing system of claim 5, wherein the mobile device is capable of maintaining a service log.

16. A method of facilitating the servicing of an ultrasound scanner by a user, the steps comprising:
positioning a near field communication enabled mobile device within a near field communication range generated by a near field communication enabled ultrasound scanner;
establishing a near field communication link between the mobile device and the ultrasound scanner when the mobile device is within the communication range;
authenticating at least one of the mobile device or the user through the established communication link; and
servicing the ultrasound scanner through the established communication link.

17. The method of claim 16, further comprising the step of generating a service report at the mobile device.

18. The method of claim 17, further comprising the step of connecting the mobile device to the internet.

19. The method of claim 18, further comprising the step of transmitting the service report to a service provider.

20. The method of claim 16, wherein the step of servicing the ultrasound scanner comprises at least one of updating software, transferring scanner configurations, collecting diagnostic data, and triggering test routines.
